(19) United States
(12) Patent Application Publication
(10) Pub. No.: US 2018/0072990 A1
(43) Pub. Date: Mar. 15, 2018

(54) GENERATING VIRUS OR OTHER ANTIGEN-SPECIFIC T CELLS FROM A NAÏVE T CELL POPULATION

(71) Applicant: CHILDREN'S NATIONAL MEDICAL CENTER, Washington, DC (US)

(72) Inventors: Catherine BOLLARD, Washington, DC (US); Conrad CRUZ, Washington, DC (US); Patrick HANLEY, Washington, DC (US)

(73) Assignee: CHILDREN'S NATIONAL MEDICAL CENTER, Washington, DC (US)

(21) Appl. No.: 15/563,854

(22) PCT Filed: Mar. 21, 2016
(86) PCT No.: PCT/US16/23413

§ 371 Date: Oct. 2, 2017

Related U.S. Application Data
(60) Provisional application No. 62/135,851, filed on Mar. 20, 2015.
(60) Provisional application No. 62/135,888, filed on Mar. 20, 2015.

(51) Int. Cl. C12N 5/0783; C07K 14/075; C07K 14/535; C07K 14/54; C07K 14/55; A61K 39/295; A61K 35/17

(52) U.S. Cl. CPC C12N 5/0636; C07K 14/075; C07K 14/535; C07K 14/5406; A61K 2039/5158; A61K 39/295; A61K 35/17; C12N 2501/59; C07K 14/55

(57) ABSTRACT

Safe, rapid and efficient methods for producing virus-specific or other antigen-specific T-cells from cord blood and other samples containing naive immune cells.

### CROSS-REFERENCE(S) TO RELATED APPLICATIONS

This application claims priority to U.S. Provisional 62/135,851, filed Mar. 20, 2015 and to U.S. Provisional 62/135,888, filed Mar. 20, 2015, the entire disclosures of which are incorporated by reference. This application is related to PCT/US2014/62698, filed Oct. 28, 2014, entitled “Expansion of CMV-Specific cells from CMV-Seronegative Donors”, which claims priority to U.S. Provisional Application No. 61/896,296, filed Oct. 28, 2013. The disclosures of all of the above-mentioned documents are incorporated by reference.

### BACKGROUND OF THE INVENTION

### Field of the Invention

The invention relates generally to the field of virus and other antigen-specific T-cells, methods for producing them from naïve T-cells and to cell-based therapy using the virus and other antigen-specific T-cells.

### Description of the Related Art

Existing T-cell based immunotherapies use virus- and tumor-specific T-cells expanded from samples containing T-cells and precursor T-cells. Virus-specific T cells have been shown to be effective against viral infections after stem cell transplant and T cell based cell therapies using virus-specific T-cell populations have been shown to provide protection from virus-infected cells and to be associated with fewer side effects than many antiviral drug therapies. T cell based therapies using expanded virus-specific populations have also demonstrated a graft-versus-leukemia effect that cleared circulating leukemic blasts. These immunotherapies have the advantage of providing lifelong protection with the generation of memory populations. Moreover, these cells are easily expanded ex vivo because the donors from which they are derived are seropositive, meaning that there are existing memory, virus-specific T cells that rapidly expand in the presence of antigen. However, these methods suffer from the requirement for T-cells obtained from a donor whose immune system already recognizes a viral- or tumor antigen (e.g., a donor who is seropositive for a particular virus), see Ngo, et al., J. Immunother. 37(4): 192-203 (2014).

When naïve T-cell or T-cell precursor population, such as those in cord blood, has never been exposed to and primed by an antigen or antigenic peptide, virus- and other antigen-specific T-cells cannot be expanded from it. Such naïve populations lack antigen-specific memory T-cells that can rapidly expand when contacted with the antigens they recognize. For example, when a subject receives a cord blood transplant, the cord blood almost entirely contains naïve T cells that do not provide protection against viruses, other pathogens or tumors. Similar transplants, such as stem cell transplants from naïve donors, such as donors seronegative for a particular virus, pathogen or tumor antigen, also lack memory T-cells that rapidly expand. Consequently, the expansion of virus-specific T cells from the cord blood or for transplants from naive donors have been limited and not clinically-applicable.

The difficulties with generating virus-specific T cells from these populations arise from: (1) the need for priming naïve antigen specific T cells, and (2) the limited volumes in umbilical cord blood. Cord blood units typically contain a total of 25 mL of blood. From this 25 mL, 20 mL typically goes directly to the patient as the transplant to repopulate the immune system, while only 5 mL is left for potential T cell expansion. Further, the naïve T cells present in the product, as well as the limited volume, have previously made this procedure implausible for the clinical setting and highlight the need for the development of new procedures for generating the kinds and numbers of virus or other antigen-specific T-cells needed for successful immunotherapy.

Existing methods for priming and expanding virus- or other antigen-specific T-cells from naïve T-cells have not been successful, see McGoldrick, et al., “Cytomegalovirus-specific T cells are primed early after cord blood transplant but fail to control virus in vivo”, Blood 121(14): 2796-2803 (Epub 2013). This is consistent with the observation that developing immune systems of neonates have little immunological memory which increases their vulnerability to infectious agents, see Basha, et al., “Immune response in neonates”, Expert Rev. Clin. Immunol. 10(9):1171-1184 (2014). Neonatal, congenital, and/or intrauterine pathogens include Rubella, Cytomegalovirus (CMV), Parvovirus B19, Varicella-Zoster (VZV), Enteroviruses, HIV, HTLV-1, Hepatitis C, Hepatitis B, Lassa Fever, and Japanese Encephalitis. Perinatal and neonatal infections agents include Herpes Simplex Virus (including Human Herpes Simplex types 1 and 2), VZV, Enteroviruses, HIV, Hepatitis B, Hepatitis C and HTLV-1. Other pathogens include respiratory syncytial virus (RSV), metapneumovirus (hMPV), rhinovirus, parainfluenza (PIV), and human coronavirus, norovirus, Herpes simplex virus (HSV), Zika virus and encephalitis viruses.

An additional problem with many existing methods for expanding virus- and other antigen-specific T-cells is that many present methods involve the user of infectious viruses, virus-infected cells, or virus-transformed cells, such as Epstein-Barr virus-transformed lymphoblastoid cell lines, Ngo, et al. (2014). Methods that involve the use of viruses to produce virus- and other antigen-specific T-cells T-cells for therapeutic use are undesirable because they are associated with increased clinical risks and significant regulatory hurdles.

One embodiment according to the invention advantageously permits the rapid and robust expansion of virus- and other antigen-specific T-cells from naïve populations thus providing virus- and other antigen-specific T-cells which recognize therapeutically important antigens, such as those of opportunistic viruses and tumor antigens. This embodiment does not require the use of live viruses or virus-transformed cells and thus is more clinically acceptable. Also it does not require the use of infectious or dangerous agents which are discouraged or prohibited by U.S. and international regulatory bodies. Moreover, the expanded T-cells according to the above embodiment can readily be used in clinical practice or can be conveniently banked and used as an off-the-shelf product.

### BRIEF SUMMARY OF THE INVENTION

In some of its embodiments, the invention provides a robust method for generating T-cells that specifically recognize particular antigens, such as those derived from viruses, other pathogens or tumors. The invention also often generates a population of T-cells that recognizes different or multiple epitopes of a pathogen providing for a broader spectrum of cellular immunity. For example, to produce a broad cellular immune response, naïve cell populations can be exposed to antigen-presenting cells pulsed with and presenting overlapping peptides representing one or more antigens of a particular pathogen, such as cytomegalovirus. These peptides may be pulsed onto different antigen presenting cells (dendritic cells, monocytes, K562 cells, PHA blasts, B-blasts, lymphoblastoid cells, and CD3-28 blasts) and the method may employ different priming and expansion cytokines (including but not limited to IL2, IL7, IL15), and different selection methods (CD45RO depletion, etc). The virus- or other antigen-specific T-cells produced by such methods can be used to treat post-transplant viral infections, infections by non-viral pathogens or tumor relapse in a subject receiving a transplant of naïve cord blood, stem or other donor cells. Moreover, the antigen-specific T-cells can be advantageously banked or stored for later administration to a subject in need of treatment, for example, in need of T-cells that recognize a particular virus or tumor.

In another embodiment, the invention provides antigen-specific T-cells, including populations of antigen-specific T-cells that recognize multiple determinants of an antigen, that can be used to boost or supplement the immune system of other subjects, including those not receiving cord blood or naïve hematological cell transplants, when needed. Examples of such subjects include those receiving organ transplants, those undergoing immune system ablation, and those who are immunosuppressed or immunocompromised, such as those infected with opportunistic infections. The invention makes multi-virus-antigen-specific T cells from naïve T cells in a clinically-relevant way that has never be done before from naïve T cells. In some embodiments, the invention itself is a process and use which can readily applied to other opportunistic viruses such as, but not limited to, HHV6 and BK viruses. It can be expanded to include virus-specific antigens from diseases associated with malignancies such as, but not limited to, those caused by or associated with EBV and HIV. Other medical uses include promoting engraftment and providing a therapy to immunodeficient patients before a transplant.

Without limitation, embodiments of the invention can be combined with other therapies, such as cellular products, lymphodepleting regimens, epigenetic-modifying drugs, or other antimicrobial or antitumor therapies.

In some embodiments the invention generates antigen-specific T cells using different overlapping peptide libraries pulsed onto different antigen presenting cells (dendritic cells, monocytes, K562 cells, PHA blasts, B-blasts, lymphoblastoid cells, and CD3-CD28 blasts), different priming and expansion cytokines (including but not limited to IL2, IL7, IL15), and different selection methods (CD45RO depletion, etc). these cells are used to treat post-transplant viral or other microbial infections.

In another embodiment the invention involves third party banking of antigen-specific T-cells manufactured from naïve T cells along with processes for selecting the best donor match.

Other advantageous features of many embodiments of the process according to the invention include that they employ simple, repeatable steps that comply with good manufacturing practices. It is not necessary to perform multiple, complex and potentially unrepeatable or non-standardizable steps. The process of the invention is safe, simple, rapid and reproducible and can be used to produce virus- and other antigen-specific T-cells for a variety of different patients.

The process according to the invention is broad in scope in that it can target different patients receiving different transplants, such as cord blood, stem cells or other naïve donor cells. For example, it is the only process that produces virus- and other antigen-specific T-cells for patients undergoing a cord blood transplant where the same cord blood unit is used for the transplant and also used to manufacture the virus and other antigen-specific T-cells that protect the patient from opportunistic infections.

Specific non-limited embodiments of the invention include the following:

1. A process for producing a virus- or other antigen-specific T cell comprising:
(a) dividing mononuclear cells from a cord blood sample or other sample containing naïve immune cells into two portions;
(b) contacting a first portion of said sample with PHA or another mitogen and/or with IL-2 to produce ATCs (“activated T cells”) and treating the ATCs with radiation or another agent to inhibit their outgrowth;
(c) separating T-cells and T-cell precursor cells (e.g., nonadherent cells, CD3⁺ cells) from dendritic cells and dendritic precursor cells (e.g., adherent cells, CD11C⁺ or CD14⁺ cells);
(d) cryopreserving or otherwise reserving the non-adherent cells;
(e) contacting the adherent cells in the second portion with cytokine(s) or other agent(s) that generate and mature dendritic cells and with at least one virus or other peptide antigen to produce antigen-presenting dendritic cells that present at least one peptide antigen, and treating said antigen-presenting dendritic cells with radiation or another agent sufficient to inhibit their outgrowth;
(f) contacting the cryopreserved or otherwise reserved non-adherent cells from (d) with the dendritic antigen-presenting cells produced in (e) in the presence of IL-7 and IL-15 to produce virus- or other antigen-specific T-cells that recognize the at least one virus antigen or other peptide antigen;
(g) contacting virus or other antigen-specific T-cells produced by (f) with the ATCs of (b) in the presence of the at least one peptide antigen, optionally, in the presence of K562 cells or other accessory cells and in the presence of IL-15; optionally, repeating (g) one or more times;
(h) recovering virus- or other antigen-specific T-cells that recognize the at least one virus- or other peptide antigen; and
(i) optionally, administering said antigen-specific T-cells to a subject in need thereof or banking or storing said antigen-specific T-cells.

2. The process of embodiment 1, further comprising separating mononuclear cells from cord blood or another sample containing naïve T-cells prior to (a).

3. The process of embodiment 1 or 2, wherein the mononuclear cells are obtained from cord blood.

4. The process of embodiment 1, 2 or 3, wherein the mononuclear cells are obtained from stem cells naïve to the at least one virus or other peptide antigen.

5. The process of embodiment 1, 2, 3 or 4, wherein the mononuclear cells are obtained from a sample containing stem cells, precursor T-cells, or T-cells from a subject whose immune system is naïve to the at least one virus or other peptide antigen.

6. The process of embodiment 1, 2, 3, 4 or 5, wherein (b) comprises contacting a first portion of said sample with PHA and with and IL-2 to produce ATCs (“activated T cells”). These ATCs may be cryopreserved or otherwise banked for later use or may be used immediately. Preferably, the ATCs are used fresh and mixed in which virus- or other antigen-specific T-cells produced in (f) without the need to cryopreserve either the ATCs or the virus- or other antigen-specific T-cells. For example, PHA blasts prepared in (b) can be used 14-16 days after initiation of the process to provide a second stimulation to the virus or other antigen-specific T-cells produced in (f).

7. The process of embodiment 1, 2, 3, 4, 5, or 6 that comprises contacting about 1 to 20 million, preferably 5-15 million, most preferably about 8-12 million, mononuclear cord blood cells with PHA and IL-2 in (b).

8. The process of embodiment 1, 2, 3, 4, 5, 6 or 7, wherein (b) comprises producing T-blasts, B-blasts, lymphoblastoid cells, or CD3-CD28 blasts.

9. The process of any one of embodiments 1-8, wherein T-cells and T-cell precursor cells are separated from dendritic cells and dendritic precursor cells by contacting the second portion with a solid medium for a time and under conditions sufficient for cells in the second portion to adhere to the solid medium and then removing T-cells and T-cell precursor cells from the solid medium and recovering the dendritic cells and dendritic precursor cells attached to the solid medium. Alternatively, these two populations of cells may be separated magnetically, by the use of antibodies or other ligands that specifically recognize each population, or by other known methods of cell sorting. The separate populations of cells may be cryopreserved or otherwise banked for later use, or may be used immediately to produce T-cells or dendritic cells. These populations may also be cryopreserved or otherwise banked after subsequent treatment steps described herein that produce mature dendritic cells loaded with virus or other peptide antigens or virus- or other antigen-specific T-cells.

10. The process of any one of embodiments 1-9, wherein in (e) the dendritic cells and dendritic precursor cells are contacted with at least one dendritic cell-generating cytokine selected from the group consisting of IL-4 and GM-CSF.

11. The process of any one of embodiments 1-10, wherein in (e) the dendritic cells and dendritic precursor cells are contacted with a dendritic cell-maturing cytokine or agent selected from the group consisting of LPS, TNF-alpha, IL-1 beta, IL-6, PGE-1 and PGE-2; along with IL-4 and GM-CSF.

12. The process of any one of embodiments 1-11, wherein in or prior to (f) the dendritic cells and dendritic precursor cells are treated to expand CD45RA positive cells.

13. The process of any one of embodiments 1-12, wherein in or prior to (f) the dendritic cells and dendritic precursor cells are treated to deplete CD45RO positive cells.

14. The process of any one of embodiments 1-13, wherein said at least one virus- or other antigen-specific peptide antigen comprises a series of overlapping peptides.

15. The process of any one of embodiment 1-14, wherein said at least one virus- or other peptide antigen comprises a tumor-associated or tumor-specific antigen.

16. The process of any one of embodiments 1-15, wherein said at least one peptide antigen comprises a determinant of a tumor-associated or tumor-specific antigen selected from the group consisting of PRAME, NYESO, MAGE A4, MAGE A3, MAGE A1, surviving WT1, neuroelastase, proteinase 3, p53, CEA, claudin6, Histone H1, Histone H2, Histone H3, Histone H4, MART1, gp100, PSA, SOX2, SSX2, Nanog, Oct4, Myc, and Ras.

17. The process of any one of embodiments 1-16, wherein said at least one peptide antigen comprises a determinant of a virus including MHC-I or MHC-II restricted virus-derived or associated peptides. Such viruses include opportunistic pathogens, emerging viral pathogens such as Zika virus, as well as other viruses associated with disease.

18. The process of any one of embodiments 1-17, wherein said at least one peptide antigen comprises a determinant of a filovirus, such as a determinant of GP, NP, VP40, VP35, VP30, or VP24 from Ebola virus.

19. The process of any one of embodiments 1-18, wherein said at least one peptide antigen comprises a determinant of a measles virus, such as a determinant of antigen P, V, C, M, N, F, P, or L.

20. The process of any one of embodiments 1-19, wherein said at least one peptide antigen is a series of overlapping peptides representing a viral antigen from an opportunistic viral pathogen, from a neonatal congenital or intrauterine pathogen, such as Rubella, Cytomegalovirus (CMV), Parvovirus B19, Varicella-Zoster (VZV), Enteroviruses, HIV, HTLV-1, Hepatitis C, Hepatitis B, Lassa Fever, and Japanese Encephalitis; or from a perinatal or neonatal pathogen such as Human Herpes Simplex, VZV, Enteroviruses, HIV, Hepatitis B, Hepatitis C, HTLV-1, Zika virus or an encephalitis virus.

21. The process of embodiments 1-20, wherein said at least one virus peptide antigen is a series of overlapping peptides representing or constituting overlapping fragments of all or part of a CMV antigen.

22. The process of any one of embodiments 1-21, wherein said at least one virus or other peptide antigen is a series of overlapping peptides representing an Epstein Barr virus (EBV) antigen or an adenovirus antigen.

23. The process of any one of embodiments 1-22, wherein said at least one virus peptide antigen comprises peptides or series of peptides from multiple viral antigens of opportunistic or emergent viral pathogens.

24. The process of any one of embodiments 1-23, wherein said at least one peptide antigen comprises a determinant of a bacterial antigen.

25. The process of any one embodiments 1-24, wherein said at least one peptide antigen comprises a determinant of a mycobacterium, such as a determinant of ESAT6, HLPMt, PPE5, MVA85A, AG85, PSTS1, ACR, HSP65, GroES, EsxA, EsxB, MPB70 from Mycobacterium tuberculosis.

26. The process of any one of embodiments 1-25, wherein said at least one peptide antigen comprises a determinant of a fungal, parasitic, or other eukaryotic pathogen.

27. The process of any one of embodiments 1-26, wherein said at least one peptide antigen comprises a mammalian histocompatibility antigen or other mammalian antigen.

28. The process of any one of embodiments 1-27, wherein in (f) the non-adherent cells from (d) are contacted with the dendritic antigen-presenting cells made in (e) at a ratio (d):(e) ranging from 1:1 to 200:1, preferably at a ratio ranging from 5:1 to 100:1, and most preferably at a ratio of about 5:1 to 20:1.

29. The process of any one embodiments 1-28, wherein (g) further comprises contacting said virus- or antigen-specific T-cells with K562 cells, modified HLA-negative, K562cs cells that express CD80, CD83, CD86, and/or 4-1BBL, or other accessory cells.

30. The process of any one of embodiments 1-29, wherein (g) comprises contacting said T-cells produced in (f) with ATCs and K568 cells at a ratio of T-cell to ATC ranging from 10:1 to 1:1, preferably ranging from 5:1 to 2:1, and most preferably at a ratio of about 4:1.

31. The process of any one of embodiments 1-30, further comprising repeating (g) with the virus- or antigen-specific T-cells recovered in (h) in the presence of IL-2.

32. A composition comprising virus- or other antigen-specific T-cells produced by the process of any one of embodiments 1-31.

33. A virus- or other antigen-specific T-cell bank comprising multiple samples of cryo- or otherwise-preserved viable virus- or other antigen-specific T-cells produced by the process of any one of embodiment 1-31.

34. A method of treatment comprising administering virus- or other antigen-specific T-cells produced by the process of any one of embodiments 1-31 to a subject in need thereof.

35. The method of embodiment 34, wherein said subject is partially histocompatible with the virus- or other antigen-specific T-cells.

36. The method of embodiment 34, wherein said subject is fully histocompatible with the virus- or other antigen-specific T-cells.

37. The method of any one of embodiments 34-36, wherein the subject's immune system has been reconstituted with the same cord blood cells or same naïve immune cells used to produce the virus- or other antigen-specific T-cells.

38. the method of any one of embodiments 34-37, wherein the subject is immunocompromised.

39. The method of any one of embodiments 34-38, wherein the subject's immune system has been ablated or lymphocyte depleted, for example by radiation, chemotherapy, infection, or immunosuppression.

40. The method of any one of embodiments 34-39, wherein the subject has received an allograft or other transplant.

41. The method of any one of embodiments 34-40, wherein the subject's immune system is naïve to the antigen recognized by the virus- or other antigen-specific T-cells produced.

42. The method of any one of embodiments 34-41, wherein the virus- or other antigen-specific T-cells recognize cytomegalovirus antigen(s) or antigenic determinants thereof or wherein the virus- or other antigen-specific T-cells recognize Epstein Barr virus antigen(s) or antigenic determinants thereof.

43. The method of any one of embodiments 34-42, wherein the virus- or other antigen-specific T-cells recognize adenovirus antigen(s) or antigenic determinants.

44. The method of any one of embodiments 34-43, wherein the virus- or other antigen-specific T-cells recognize multiple antigens or antigenic determinants of one or more opportunistic viral pathogen(s).

45. The method of any one of embodiments 34-44, wherein the virus-specific T-cells recognize at least one virus antigen of an opportunistic viral pathogen selected from the group consisting of CMV, adenovirus, BK virus, Human Herpes Virus-6 (HHV6) or other herpes viruses, influenza, respiratory syncytial virus, parainfluenza virus, and Varicella Zoster virus.

46. The method of any one of embodiments 34-45, wherein the virus- or other antigen-specific T-cells recognize at least one antigen of an opportunistic viral pathogen that is acquired nosocomially or iatrogenically or that is transmitted to a subject in a hospital (e.g., a hospital acquired infection).

47. A composition comprising mononuclear cells isolated from cord blood or from another sample containing naïve immune cells, PHA or another mitogen, IL-2 and a medium that maintains the viability of said cells, and, optionally, K562 cells or other non-autologous cells that costimulate T-cells, wherein, optionally, said cells have been treated to prevent outgrowth.

48. A composition comprising:
(j) T-cells and T-cell precursor cells (e.g., nonadherent cells, CD3⁺ cells) that have been separated from dendritic cells and dendritic precursor cells (e.g., adherent cells, CD11C⁺ or CD14⁺ cells),
(ii) IL-7 and IL-15, and
(iii) a medium that maintains the viability of said T-cells and T-cell precursor cells.

49. The composition of any one of embodiments 47-48, wherein the mononuclear cells, T-cells or T-cell precursor cells have been contacted with dendritic cells that have been contacted or pulsed with at least one peptide antigen, and wherein said composition comprises mononuclear cells, T-cells or T-cell precursor cells that recognize the at least one peptide antigen.

50. A composition comprising dendritic cells and dendritic precursor cells (e.g., adherent cells, CD11C⁺ or CD14⁺ cells) that have been separated from T-cells and T-cell precursor cells (e.g., non-adherent cells, CD3⁺ cells), at least one agent that generates and matures dendritic cells, and a medium that maintains the viability of said cells; wherein, optionally, said cells have been contacted with one or more peptide antigens and, optionally, treated to prevent outgrowth.

51. A bank or cell storage facility which contains one or more samples of the compositions according to any of embodiments 47-50 in combination with a storage or freezing medium; wherein said one or more samples is optionally associated, identified or indexed by information describing its source, including full or partial DNA sequence information, information describing its histocompatibility, such as information describing at least one major and/or minor histocompatibility antigen or marker, and/or information about the peptide antigens it contains or recognizes.

### BRIEF DESCRIPTION OF THE DRAWINGS

The figures describe particular, non-limiting embodiments of the invention.

FIG. 1. Dendritic cell, PHA blast initiation, and cryopreservation of non-adherent cells.

FIG. 2. Dendritic cell maturation and pulsing with peptide antigens.

FIG. 3. 1^{st} T-cell stimulation with dendritic cells.

FIG. 4. 2^{nd} and subsequent T-cell stimulations.

FIG. 5. A general description of one embodiment of the invention.

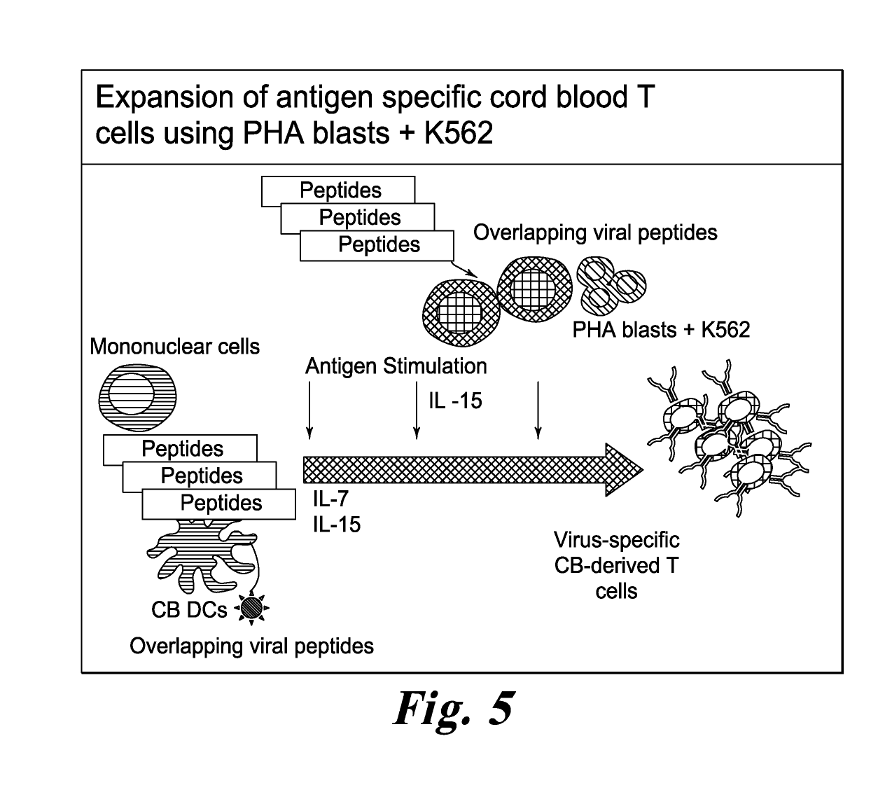

### DETAILED DESCRIPTION OF THE PREFERRED EMBODIMENTS

“Accessory cell” is a cell, such as a K562 cell, that provides costimulation for recognition of peptide antigens by T-cells or that otherwise assists a T-cell recognize, become primed or expand in the presence of a peptide antigen.

An “activated T-cell” or “ATC” according to the invention is obtained by exposing mononuclear cells in cord blood or another sample containing naïve immune cells to a mitogen, such as Phytohemagglutinin (PHA) and Interleukin (IL)-2.

An “antigen” includes molecules, such as polypeptides, peptides, or glyco- or lipo-peptides that are recognized by the immune system, such as by the cellular or humoral arms of the human immune system. the term “antigen” includes antigenic determinants, such as peptides with lengths of 6, 7, 8, 9, 10, 11, 12, 13, 14, 15, 16, 17, 18, 19, 20, 21, 22 or more amino acid residues that bind to MHC molecules, form parts of MHC Class I or II complexes, or that are recognized when complexed with such molecules.

An “antigen presenting cell (APC)” refers to a class of cells capable of presenting one or more antigens in the form of peptide-MHC complex recognizable by specific effector cells of the immune system, and thereby inducing an effective cellular immune response against the antigen or antigens being presented. Examples of professional APCs are dendritic cells and macrophages, though any cell expressing MHC Class I or II molecules can potentially present a peptide antigen.

A “control” is a reference sample or subject used for purposes of comparison with a test sample or test subject. Positive controls measure an expected response and negative controls provide reference points for samples where no response is expected.

“Cord blood” has its normal meaning in the art and refers to blood that remains in the placenta and umbilical cord after birth and contains hematopoietic stem cells. Cord blood may be fresh, cryopreserved or obtained from a cord blood bank.

The term “cytokine” has its normal meaning in the art. Examples of cytokines used in the invention include IL-2, IL-7 and IL-15.

The term “dendritic cell” or “Dc describes a diverse population of morphologically similar cell types found in a variety of lymphoid and non-lymphoid tissues, see Steinman, Ann. Rev. Immunol. 9:271-296 (1991). One embodiment of the invention involves dendritic cells and dendritic cell precursors derived from cord blood.

The term “effector cell” describes a cell that can bind to or otherwise recognize an antigen and mediate an immune response. Virus- or other antigen-specific T-cells are effector cells.

The term “isolated” means separated from components in which a material is ordinarily associated with, for example, an isolated cord blood mononuclear cell can be separated from red blood cells, plasma, and other components of cord blood.

A “naive” T-cell or other immune effector cell is one that has not been exposed to or primed by an antigen or to an antigen-presenting cell presenting a peptide antigen capable of activating that cell.

A “peptide library” or “overlapping peptide library” within the meaning of the application is a complex mixture of peptides which in the aggregate covers the partial or complete sequence of a protein antigen, especially those of opportunistic viruses. Successive peptides within the mixture overlap each other, for example, a peptide library may be constituted of peptides 15 amino acids in length which overlapping adjacent peptides in the library by 11 amino acid residues and which span the entire length of a protein antigen. Peptide libraries are commercially available and may be custom-made for particular antigens. Methods for contacting, pulsing or loading antigen-presenting cells are well known and incorporated by reference to Ngo, et al. (2014). Peptide libraries may be obtained from JPT and are incorporated by reference to the website at https://www.jpt.com/products/peptrack-peptide-libraries/ (last accessed Mar. 21, 2016).

The term “precursor cell” refers to a cell which can differentiate or otherwise be transformed into a particular kind of cell. For example, a “T-cell precursor cell” can differentiate into a T-cell and a “dendritic precursor cell” can differentiate into a dendritic cell.

A “subject” is a vertebrate, preferably a mammal, more preferably a human. Mammals include, but are not limited to humans, simians, equines, bovines, porcines, canines, felines, murines, other farm animals, sport animals, or pets. Subjects include those in need of virus- or other antigen-specific T-cells, such as those with lymphocytopenia, those who have undergone immune system ablation, those undergoing transplantation and/or immunosuppressive regiments, those having naïve or developing immune systems, such as neonates, or those undergoing cord blood or stem cell transplantation.

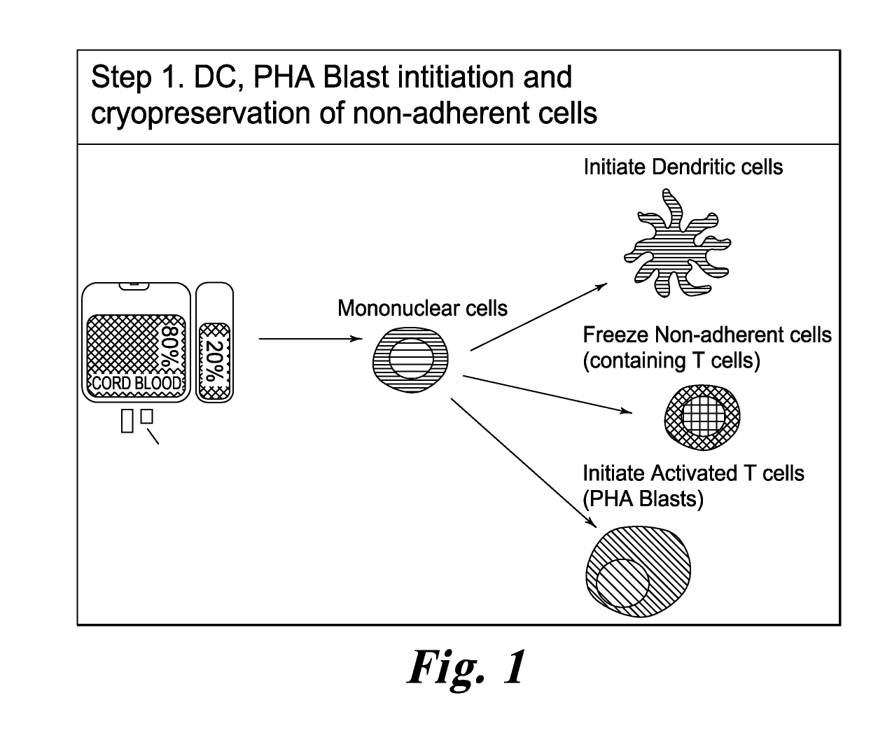

In one nonlimiting embodiment of the invention, cord blood is used to produce the virus- or other antigen-specific T-cells as described by FIGS. 1, 2, 3 and 4 and as explained in more detail below.

Step 1. As shown in FIG. 1, cord blood unit is processed to isolate the mononuclear cells (MNC). From the MNC three subsets were isolated and expanded: 1) the immature dendritic cells (DCs), which are isolated by plastic adherence, 2) the T cell-containing fraction, the non-adherent cells, which are cryopreserved for later use, and 3) PHA blasts, which are non-specifically activated T cells that are used later as antigen presenting cells. These are generated from ˜5 million MNC. Once adherent, the adherent cells (DCs) are fed with IL-4 and GM-CSF. This method is novel in that the PHA blasts are generated from the starting product (which is typically cryopreserved).

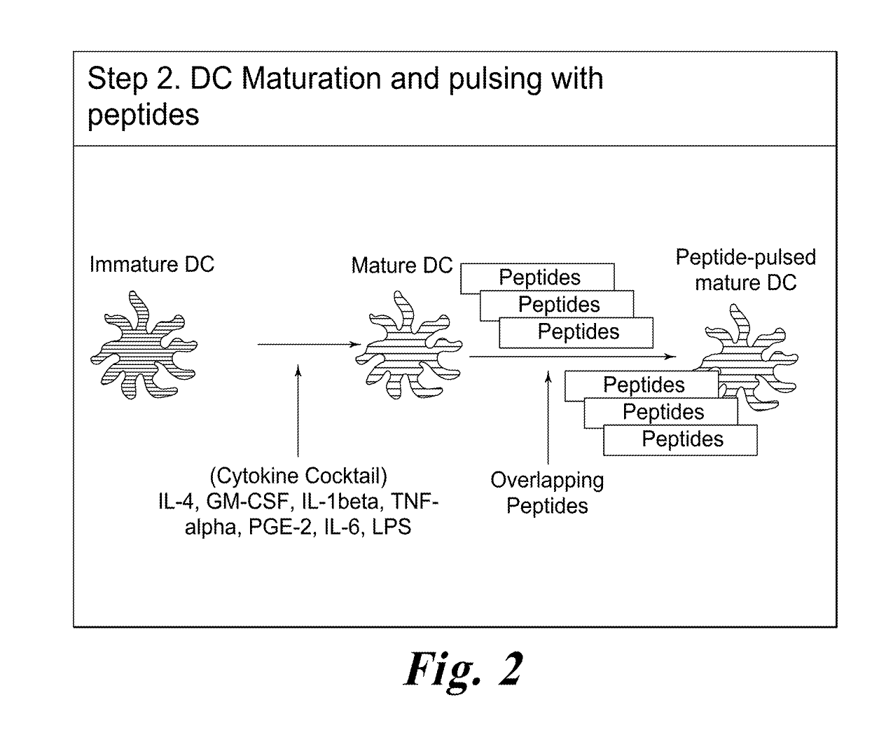

Step 2. As shown in FIG. 2, about 5 days after initiation, the dendritic cells are matured by adding a cytokine cocktail containing IL-4, GM-CSF, IL-1beta, TNF-alpha, PGE-2, IL-6, and LPS. LPS is novel in this application. From the peripheral blood setting the use of adherence for DCs is also different (they use CD14-selection to enrich for DC precursors).

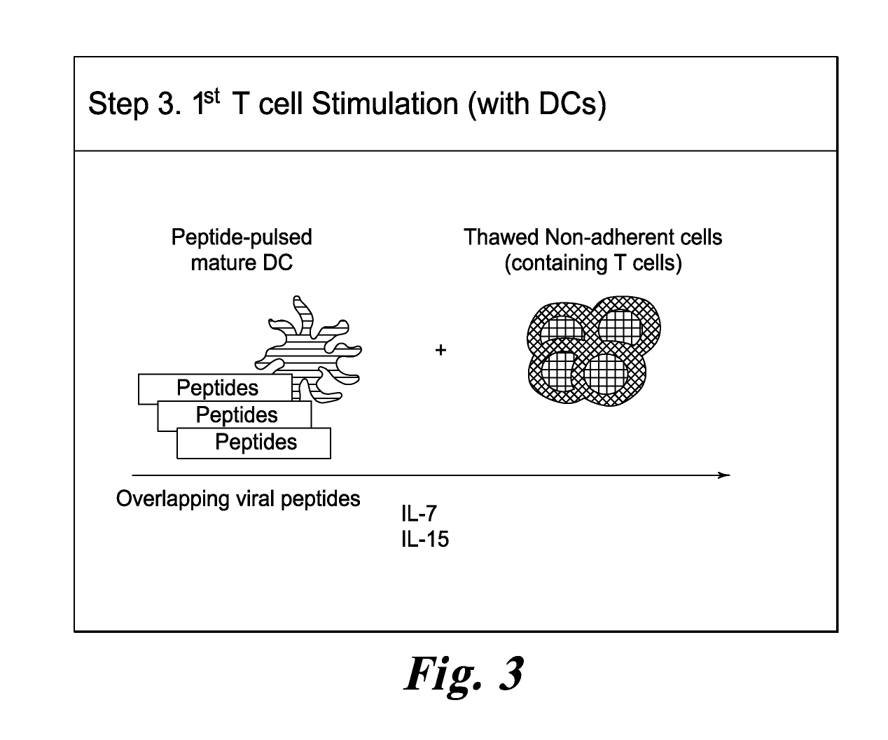

In step 3, as shown in FIG. 3, at initiation, the matured dendritic cells are pulsed with overlapping peptides, irradiated so that they do not expand, and they are then combined with the non-adherent cells (which are thawed in the presence if IL-7 and IL-15. IL-12 is no longer used.

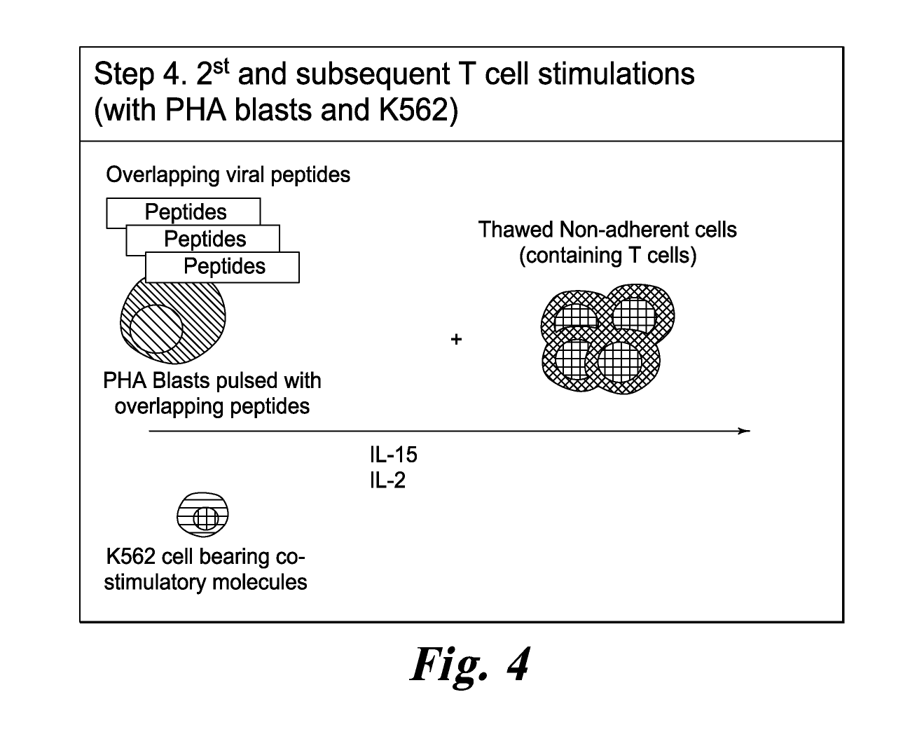

In step 4 as shown in FIG. 4, which is about 14-16 days from initiation of the culture (7-9 days from the first T cell stimulation), PHA blasts (derived from the same cord blood are pulsed with the same overlapping peptides, irradiated, and then combined with K562 cells; the combination of these two act as the antigen-presenting cells for the previously-expanded T cells. The use of the peptide-pulsed PHA-blasts and K562 differs from previous cord blood generation protocols. In this embodiment, advantageously the T cells do not need to be frozen after one expansion. Prior methods required one to wait for the LCL to be ready before continuing. Since no waiting for the LCL is required, the antigen-specific T-cells can be manufactured in about 30 days instead of 60. Another difference with prior methods is that PHA blasts are used instead of CD3/CD28 blasts and because T cells responding to the PHA are naïve T cells, unlike in prior protocols which used peripheral blood where the majority of T-cells were memory cells.

### EXAMPLE

### Production and Expansion of Virus- or Other Antigen-Specific T Cells From Cord Blood

Non-adherent mononuclear cells (e.g., naïve T cells) isolated from cord blood were simulated by contact with irradiated peptide-pulsed antigen presenting cells prepared from non-adherent cells (e.g., monocytes, dendritic cells, etc.) in cord blood and then by irradiated peptide-pulsed antigen presenting cells non-specifically expanded from cord blood. This method was produced virus- or other antigen specific T-cells from cord blood cells.

Specifically, mononuclear cells were isolated from cord blood by centrifugation at 800×g for 20 minutes with little acceleration and brake and at room temperate on a Ficoll gradient. Approximately 10 million of the isolated mononuclear cells were reserved to produce non-specifically expanded T cells (antigen-presenting cells) also known as “Activated T Cells” or “ATCs”. In this case, Phytohemagglutinin (PHA) was used to stimulate the ATCs.

The remaining isolated mononuclear cells were plated onto tissue culture plates containing Cellgenix CellGro serum-free medium. After 1-2 hours, the tissue culture plates was washed with PBS to remove non-adherent cells which were then cryopreserved and saved for later use.

The cells that adhered to the cell culture plates after washing were mixed with cytokines to generate dendritic cells (DC). This was done by contacting the cells with 1000 U/mL Interleukin (IL)-4, and 800 U/mL Granulocyte-Macrophage/Colony Stimulating Factor (GM-CSF) and then with 30 ng/mL Lipopolysaccharide (LPS), 10 ng/mL Tumor Necrosis Factor Alpha (TNF-α), 10 ng/mL IL-1β, 100 ng/mL IL-6, and 1 ug/mL Prostaglandin (PGE)-2 or PGE-1, along with 1000 U/mL IL-4 and 800 U/mL GM-CSF.

Once the dendritic cells matured for 7 days from initiation and they were pulsed with a pool of overlapping peptides containing about 200 ng of each peptide per million cells obtained from an overlapping peptide library. In this case we used the overlapping peptides from JPT including IE-1 and pp65 from CMV, Hexon and Penton from Adenovirus, and LMP2 and BZLF-1 from EBV. These overlapping peptide mixtures, or “Pepmixes,” consist of 15 amino acid peptides that span the entire protein (antigen) and overlap neighboring peptides by 11 amino acids. This allows for the expansion of both CD4+ and CD8+ T cells, regardless of the MHC class-restriction. Following the pulsing of the mature dendritic cells with the pool of overlapping peptides the cells were irradiated at 25 Gy to prevent their outgrowth.

At this time, the cryopreserved non-adherent cells previously washed off the cell culture plates were thawed and plated with the peptide-pulsed dendritic cells at an approximate ratio of 1 DC to 10 non-adherent cells in the presence of the cytokines 10 ng/mL IL-7 and 5 ng/mL IL-15. This represented an initial antigen-stimulation of the cryopreserved non-adherent mononuclear cells (e.g., naïve T cells). Cells were grown in a naïve T cell-specific medium containing 45% Advanced RPMI, 45% Click's (EHAA) medium, 10% human AB serum, and 200 mM Glutamax.

The cryopreserved non-adherent cells were cultured for 8-10 days in the presence of the irradiated (25 Gy for DC, 75 Gy for ATCs and K562) peptide-pulsed non-adherent cells (e.g., naive T cells) and then harvested, the number of T-cells determined, and resuspended in a T cell medium.

The T-cells in the resuspension were contacted with irradiated ATCs, which have been pulsed with the same pool of overlapping peptides that were present on the irradiated mature dendritic cells derived from the adherent mononuclear cells of cord blood, at a ratio of 1 T-Cells to 1 irradiated ATC to 5 K562 cells in the presence of cytokine IL-15 (5 ng/mL) followed by twice-weekly feeds with the IL-2 cytokine (50-100 U/mL). After this secondary stimulation, T-cells which recognized antigenic determinants in the pool of overlapping peptides were recovered. This was achieved by assessing T cell activation via IFN-gamma ELISPOT assay and assessing the cytolytic ability of the T cells in a chromium release cytotoxicity assay.

All publications and patent applications mentioned in this specification are herein incorporated by reference to the same extent as if each individual publication or patent application was specifically and individually indicated to be incorporated by reference.

The invention now being fully described, it will be apparent to one of ordinary skill in the art that many changes and modifications can be made thereto without departing from the spirit or scope of the following claims.

1. A process for producing a virus- or other antigen-specific T cell comprising:
(a) dividing mononuclear cells from a cord blood sample or other sample of naïve immune cells into two portions;
(b) contacting a first portion of said sample with PHA or another mitogen and, optionally with IL-2, to produce ATCs (“activated T cells”) and treating the ATCs with radiation or another agent to inhibit their outgrowth;
(c) separating non-adherent T-cells and T-cell precursor cells from adherent dendritic cells and dendritic precursor cells);
(d) cryopreserving or otherwise reserving the non-adherent cells;
(e) contacting the adherent cells in the second portion with IL-4 and GM-CSF or other cytokine(s) and/or other agent(s) that generate and mature dendritic cells and with at least one peptide antigen to produce antigen-presenting dendritic cells that present the at least one peptide antigen, and treating said antigen-presenting dendritic cells with radiation or another agent sufficient to inhibit their outgrowth;
(f) contacting the cryopreserved or otherwise reserved non-adherent cells from (d) with the dendritic antigen-presenting cells produced in (e) in the presence of IL-7 and IL-15 to produce antigen-specific T-cells that recognize the at least one peptide antigen;
(g) contacting the antigen-specific T-cells produced by (f) with the ATCs of (b) in the presence of the at least one peptide antigen in the presence if K562 cells or other accessory cells and in the presence of IL-15; optionally, repeating (g) one or more times;
(h) recovering the antigen-specific T-cells that recognize the at least one peptide antigen; and
(i) optionally, administering said antigen-specific T-cells to a subject in need thereof or banking or storing said antigen-specific T-cells.

2. The process of claim 1, further comprising separating mononuclear cells from cord blood or another sample containing naïve T-cells prior to (a).

3. The process of claim 1, wherein the mononuclear cells are obtained from cord blood.

4. The process of claim 1, wherein the mononuclear cells are obtained from stem cells naïve to the at least one peptide antigen.

5. The process of claim 1, wherein the mononuclear cells are obtained from a sample containing stem cells, precursor T-cells, or T-cells from a subject whose immune system is naïve to the at least one peptide antigen.

6. The process of claim 1, wherein (b) comprises contacting a first portion of said sample with PHA and with and IL-2 to produce ATCs (“activated T cells”).

7. The process of claim 1 that comprises contacting about 1 to 20 million mononuclear cord blood cells with PHA and IL-2 in (b).

8. The process of claim 1, wherein (b) comprises producing T-blasts, B-blasts, lymphoblastoid cells, or CD3-CD28 blasts.

9. The process of claim 1, wherein T-cells and T-cell precursor cells are separated from dendritic cells and dendritic precursor cells by contacting the second portion with a solid medium for a time and under conditions sufficient for cells in the second portion to adhere to the solid medium and then removing non-adherent T-cells and T-cell precursor cells from the solid medium and recovering the dendritic cells and dendritic precursor cells attached to the solid medium.

10. The process of claim 1, wherein in (e) the dendritic cells and dendritic precursor cells are contacted with at least one dendritic cell-generating cytokine selected from the group consisting of IL-4 and GM-CSF.

11. The process of claim 1, wherein in (e) the dendritic cells and dendritic precursor cells are contacted with a dendritic cell-maturing cytokine or agent selected from the group consisting of LPS, TNF-alpha, IL-1 beta, IL-6, PGE-1 and PGE-2; along with IL-4 and GM-CSF.

12. The process of claim 1, wherein in or prior to (f) the dendritic cells and dendritic precursor cells are treated to expand CD45RA positive cells.

13. The process of claim 1, wherein in or prior to (f) the dendritic cells and dendritic precursor cells are treated to deplete CD45RO positive cells.

14. The process of claim 1, wherein said at least one peptide antigen comprises a series of overlapping peptides.

15. The process of claim 1, wherein said at least one peptide antigen comprises a tumor-associated or tumor-specific antigen.

16. (canceled)

17. The process of claim 1, wherein said at least one peptide antigen comprises a virus antigen.

18.-28. (canceled)

29. The process of claim 1, wherein (g) further comprises contacting said peptide antigen-specific T-cells with K562 cells, modified HLA-negative, K562cs cells that express CD80, CD83, CD86, and/or 4-1BBL cells.

30. (canceled)

31. The process of claim 1, further comprising repeating (g) with the peptide antigen-specific T-cells recovered in (h) in the presence of IL-2.

32. An in vitro composition comprising antigen-specific T-cells produced by the process of claim 1.

33. (canceled)

34. A method of treatment comprising administering antigen-specific T-cells produced by the process of claim 1 to a subject in need thereof.

35.-51. (canceled)
